(19) United States
(12) Patent Application Publication
(10) Pub. No.: US 2007/0244425 A1
(43) Pub. Date: Oct. 18, 2007

(54) Irrigation and aspiration handpiece device

(76) Inventor: Gary J. Pond, Racine, WI (US)

Correspondence Address: RYAN KROMHOLZ & MANION, S.C., POST OFFICE BOX 26618, MILWAUKEE, WI, 53226, US

(21) Appl. No.: 11/728,821

(22) Filed: Mar. 27, 2007

Related U.S. Application Data
(60) Provisional application No. 60/786,958, filed on Mar. 29, 2006.

(51) Int. Cl. A61M 1/00

(52) U.S. Cl. 604/27

(57) ABSTRACT

A needle assembly for dispensing fluids from a fluid source and for evacuating a cavity during an endodontic procedure and a method of using the needle assembly. The needle comprises a housing and a surgical needle in fluid communication with said fluid source. The housing comprises a first pathway containing said surgical needle, and a second evacuation pathway providing evacuation means for the needle assembly. The evacuation pathway is independent and external of the surgical needle.

### RELATED APPLICATION

The present invention claims priority to co-pending provisional application, U.S. Ser. No. 60/786,958, filed 29 Mar. 2006.

### BACKGROUND OF THE INVENTION

This invention relates to multipurpose dental handpieces, and in particular, handpieces which irrigate and aspirate during endodontic procedures, such as root canal surgery. During endodontic procedures, such as root canals, it is necessary to inject or applicate fluid into the dental pulp or root. Additionally, debris and other matter must be removed from the dental cavity. Typically handpiece designs for these types of procedures spray fluid, under positive pressure, into the tooth cavity. This arrangement has been known to cause a number of difficulties, most notably damage to the tooth cavity caused by undue fluid force.

During a typical root canal procedure, a dental practitioner drills an opening in a patient's tooth surface enamel and inner dentine to gain access to the dental pulp and surrounding cavity. A hollow, surgical needle is inserted into the opening to both remove decaying pulp tissue by aspiration, and irrigate the cavity with a sodium hypochlorite solution. The sodium hypochlorite solution rids the canal of bacteria and other foreign substances before sealant is injected into the canal. The dental pulp cavity is curvately elongate and tapers into the root area of the affected tooth. Common previous dental practices included the use of a handpiece fitted with a rigid, stainless steel needle whereby the practitioner alternatively aspirates and irrigates the canal. Several problems are encountered with this arrangement. First, since a stainless steel needle is relatively rigid with respect to the tooth canal and cavity, care must be taken not to puncture the tooth wall and surrounding jaw. Further, access to the extreme distal end of the curved root cavity is not possible due to the rigid nature of the needle. Additionally, the force by which fluid is discharged through the needle can create undue pressure on the tooth apex, tooth walls, and surrounding periradicular tissue, which can lead to apical extrusion accidents, particularly when irrigating the tooth with sodium hypochlorite, making full aspiration and irrigation of the canal without damage extremely difficult.

The prior art has made improvements to this process to provide both irrigation and aspiration in the same device. U.S. Pat. No. 3,624,907, issued to Brass et al., describes a device that provides both irrigation and aspiration within the same device. However, the device does not provide adequate portability and flexibility in that it does not allow for sufficient independent aspiration from the irrigation pathway. It also does not contemplate a device that includes a portable fluid source. That is, the device does not allow for hook-up to a separate vacuum source for aspiration, which limits where and how the device may be used during actual procedures. U.S. Pat. No. 6,464,498, issued to Pond, describes a irrigation and aspiration device where irrigation is supplied through gravity and surface tension on the outside of an aspiration needle. While the device provides both irrigation and aspiration capabilities within the same device, the device may be improved upon, specifically with improving fluid flow through the device, especially flow of fluids through an aspiration device. Because the aspiration flow may contain solid bits, a needle may get clogged when removing such solid-containing fluids. Likewise, the flow of solution along the exterior of the needle may not necessarily deliver the most desirable fluid flow for a specific procedure. Thus, the present invention is contemplated to overcome the limitations of the prior art.

### SUMMARY OF THE INVENTION

It is therefore an object of this invention to provide a needle assembly to be used with an endodontic handpiece or another dental device, such as a syringe, which is capable of irrigating a root canal safely and accurately, while additionally providing aspiration. The aspiration and irrigation are provided by independent sources, which allows the device to have added portability over the prior art. The needle assembly of the invention provides a dental tool with means for irrigation and means for aspiration, wherein irrigation is supplied through a needle and aspiration is supplied on the exterior surface of the needle or another location in the needle assembly that is external of the needle. This arrangement lessens the unwanted effects of clogging and uneven fluid flow during aspiration, while providing the convenience of a dual purpose tool. The needle assembly will preferably be used in connection with a control mechanism disposed on the handpiece of the dental tool, which controls whether fluid dispenses from a fluid discharge nozzle, aspirating vacuum is supplied to an aspiration nozzle, or a combination of irrigation and evacuation is performed. The assembly may also have means to transmit a physiologically safe electrical voltage or current in conjunction with the delivery of the fluid, to thereby stimulate and enhance the efficacy of the fluid. Similarly, the assembly may provide means for heating the fluid or applying a special light intense source in the system, which will also increase the efficacy of the system.

The needle assembly may further be an autoclavable endodontic needle assembly capable of curving to the configuration of a root canal while being inserted therein, or it may be a disposable assembly. The needle of the present assembly may be produced to be pre-bent to a desired angle; the preferred angle chosen is approximately 90 degrees, but other angles and straight assemblies are contemplated. The needle of the present invention may also be provided with an angle-adjustment sleeve around a portion of the needle to allow for manual adjustment of the pre-bent angle. The assembly may also be disposable.

### BRIEF DESCRIPTION OF THE DRAWINGS

FIG. 1 is a perspective view of the needle assembly of the present invention shown in connection with a dental handpiece.

FIG. 2 is partially cut-away side view of the arrangement in FIG. 1, with the cut-away section taken along line 2-2 of FIG. 1.

FIG. 3 is an overhead planar view of the present invention.

FIG. 4 is a cross-sectional side view of the device of FIG. 3.

FIG. 5 is an elevation side view of the device shown in FIG. 3.

FIG. 6 is an overhead planar view of an alternate embodiment of the present invention.

FIG. 7 is a cross-sectional view of the embodiment of FIG. 6.

FIG. 8 is an elevation side view of the embodiment of FIG. 6.

FIG. 9 is a perspective view of a device according to the present invention attached to a syringe.

FIG. 10 is an exploded view of the device shown in FIG. 9.

### DESCRIPTION OF THE PREFERRED EMBODIMENT

Although the disclosure hereof is detailed and exact to enable those skilled in the art to practice the invention, the physical embodiments herein disclosed merely exemplify the invention which may be embodied in other specific structures. While the preferred embodiment has been described, the details may be changed without departing from the invention.

The term “fluid”, as used herein, shall be defined as a gas, a liquid, a substance which flows, or a substance which differs from a solid in that it can offer no permanent resistance to change of shape. It shall further include mixtures of gases, mixtures of liquids, and mixtures of gases and liquids.

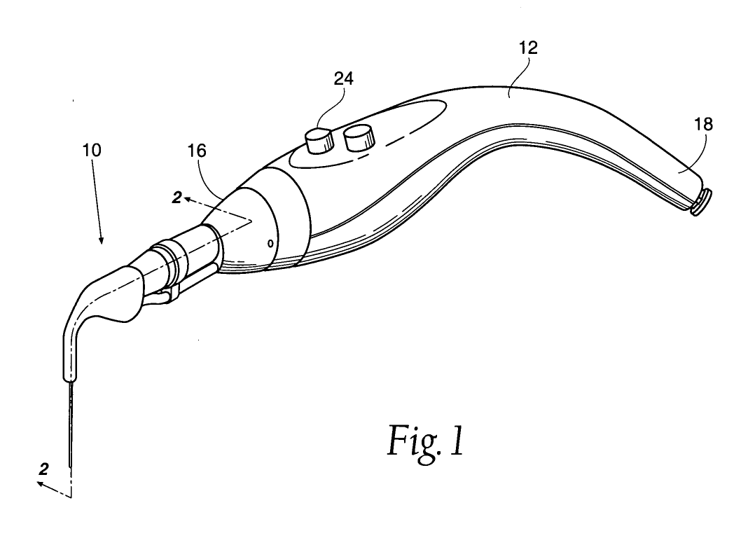

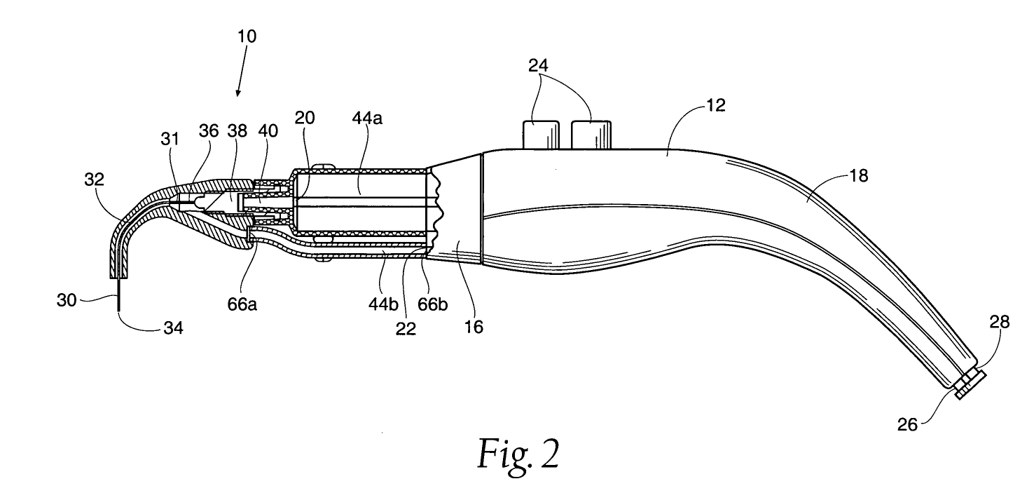

With reference to FIG. 1, a needle assembly 10 capable of dispensing fluids to and evacuating a cavity (not shown) during endodontic procedures is seen. The assembly 10 is attached to a handpiece 12. It should be understood that the handpiece 12 is merely exemplary of any of several dental devices that the needle assembly 10 can be used in connection with and should not be considered as limiting on the scope of the present invention. The handpiece 12 includes a distal end 16 and a proximal end 18, the proximal end 18 being adapted for facile gripping by a dental practitioner. As seen in FIG. 2, the distal end 16 includes a fluid discharge nozzle 20 and an aspiration nozzle 22. A control mechanism or mechanisms, seen as manual switches 24, is disposed on the handpiece 12. The control mechanism 24 controls whether fluid dispenses from the fluid discharge nozzle 20, aspirating vacuum is supplied to the aspiration nozzle 22, or a combination of irrigation and evacuation is performed. The switch 24 may also be provided with a fingerless lock-on feature that permits the switch to be maintained in an “on” position without the need for constant finger operation by the user. Alternatively, the handpiece may be provided with separate switches 24 for each aspiration or irrigation function. At least one fluid inlet 26 and one fluid outlet 28 (see FIG. 2) are each disposed, preferably at the proximal end 18 of the handpiece 12.

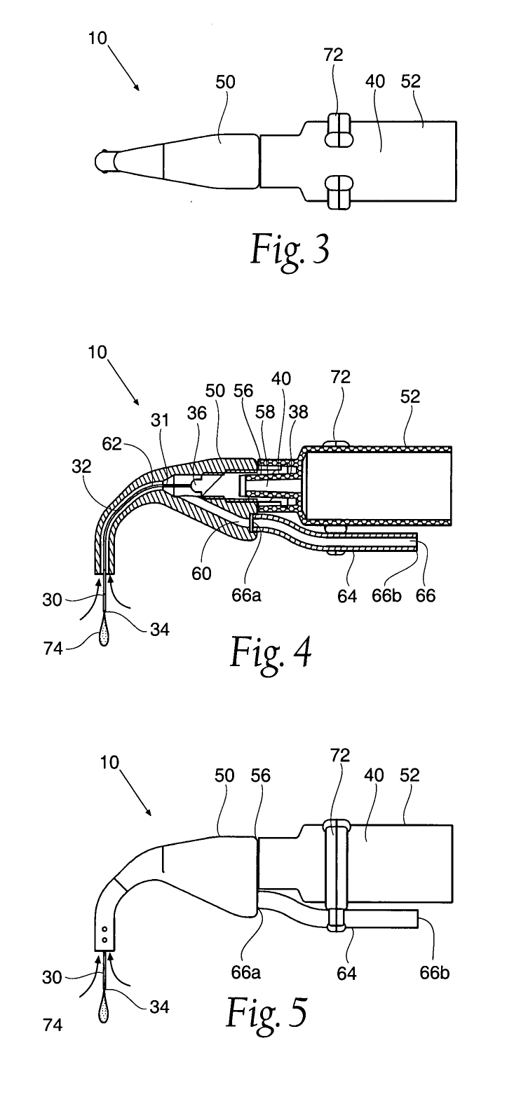

The needle assembly 10 is preferably a disposable assembly, but could also be an autoclavable endodontic needle assembly, if desired. As seen particularly in the views of FIGS. 3-5, the needle assembly 10 includes a surgical needle 30 having a shaft 31 including a hollow bore 32, a tip portion 34 and an end attachment portion 36. The needle 30 provides a fluid pathway for irrigation purposes. The needle 30 is preferably mounted on a hub member or hub apparatus 38. The hub apparatus 38 is preferably provided with a cup-like interior and is arranged for mating arrangement with a conventional LUER® connector 40 or similar connecting configuration. The connector 40 is also commonly referred to as a slip LUER® or a LUER® lock fitting. The hub apparatus 38 can be molded from an autoclavable material such as Ultim 1000, which is manufactured by the General Electric Corporation, or other materials such as polypropylene plastic, but can be composed of any material commonly used in the dental industry.

The needle 30 is preferably fabricated from a material that is corrosion resistant that may also have a flexibility incorporated into the needle. One example is a stainless steel material. Other potential materials include NiTi alloys, nylon material, or other suitable material, whereby the needle 30 is capable of curving to the configuration of a root canal while being inserted therein. The needle 30 of the present needle assembly 10 may be produced to be pre-bent to a desired angle; the preferred angle chosen is about 90 degrees, but any desired angle design is applicable. The needle 30 of the present invention may also be provided with an angle-adjustment sleeve (not shown). The angle adjustment sleeve allows for manual adjustment of the pre-bent angle.

It is to be further noted that use of sodium hypochlorite solution, or other similar performing solutions, as an irrigant can be caustic and have an adverse affect on the material used for the needle 30. To substantially eliminate the possibility of the solution corroding or deteriorating the needle 30, a coating (not seen in these views), such as a parylene polymer, may be applied to the needle 30 during its manufacture. While parylene polymers are the preferred coatings, there are other commercially available coatings that provide similar protection. The coating inhibits the sodium hypochlorite solution or similar performing solution from adversely affecting the physical properties of the dental needle 30.

The tip portion of the needle 30 may be of any shape and arrangement that will allow delivery of an irrigation fluid. For instance, the tip portion 34 may be flat, angled, skived, have several holes, or other known needle tip arrangements. The tip portion allows easy delivery of an irrigation fluid during a dental procedure, while minimizing the problems of spraying associated with the prior art. The tip 34 is also further capable of functioning within the narrow and curved confines of a root canal.

As seen in FIG. 2, the handpiece 12 is preferably adapted to contain two fluid passageways 44a, 44b. The first passageway or fluid draw line 44a provides a pathway for fluid moving from a fluid source (not shown) to the discharge nozzle 20. Once again the use of the handpiece 12 is merely exemplary, and other devices or arrangements may be used in combination with the needle assembly 10. The second or evacuation passageway 44b provides a pathway for evacuation of fluid and debris from the distal end or tip 34 of the needle 30 to an outlet reservoir or vacuum source (not shown). The second passageway 44b may be found completely within the handpiece 12, running along the outside of the handpiece 12 and, may be designed to be a stand alone element that would be connected to a suction supply that is separate from the handpiece or dental device 12. Any of these arrangements will fall within the scope of the present invention.

As seen more particularly in FIGS. 3-5, the needle assembly 10 of the present invention comprises a housing 50 which is connected to a mating section 52. It should be noted that the mating section 52 is not necessary, but could be used as a further adaptor for connecting the housing to differing dental devices. For example, in the handpiece 12, the area that would be considered the mating section is integrally made with the handpiece 12. The mating section 52 provides means for connecting the assembly 10 to a dental device, such as the handpiece 12, and can be designed as an individual component or be integrally formed with the handpiece 12 or other dental device. The connection means provided by the mating section 52 will be of any suitable shape or arrangement that will allow the assembly 10 to be attached to a specific dental device. That is, if the assembly 10 is to be attached to a handpiece, the mating section 52 will be adapted so the needle assembly 10 may be attached to the handpiece, and if the assembly 10 is to be attached to a syringe, the mating section 52 will be adapted so the needle assembly 10 will be attached to the syringe. As previously stated, the assembly 10 can be used in connection with various devices and should not be limited to use with a single device.

The housing 50 comprises a general connector area 56 that has a first passageway 58 adapted to receive the needle 30 and the hub connector 38. Also within the housing 50 is a second evacuation passageway 60 that is connected to a through bore 62 to allow a pathway for aspiration to take place with the assembly 10. It should be noted that reference to a first passageway or a second passageway is only to differentiate the two passageways and should not limit the structure or arrangement of the device or to limit the function that each passageway may provide. Provided that there is an evacuation passageway and an irrigation pathway, the arrangement will be considered as being within the scope of the present invention. The supporting hub member 38 preferably contains an adhesive material which grippingly engages the needle shaft 32 to provide connection to a conventional LUER® lock 40, but other fittings, such as slip tip fittings and eccentric fittings may also be employed. The needle 30 is positioned in the through bore 62 and through the first passageway 58. Additional adhesive (not shown) may be used to seal the area around the needle attachment end 36. The LUER® lock 40 is adapted to be received by the discharge nozzle 20 (seen in FIG. 2), thereby completing the connection from discharge nozzle 20 to needle tip 34 by way of the connector 56. It is also possible to design the first passageway 58 to accommodate the hub member 38 in a press fit type of arrangement that prevent fluids from leaking and provides a through passage to travel from the handpiece 12 or other dental device.

The second passageway 60 of the connector 56 is adapted to receive a flexible tubing length 64 having a through-bore 66. This is similar to the second passageway 44b in FIG. 2. As best viewed in FIGS. 2 and 5, the flexible tubing length 64 includes two ends 66a, 66b. The first end 66a is fittingly secured to the second passageway 60 of the connector 56, while the second end 66b fits over the aspiration nozzle 22 of handpiece 12 or another evacuation device. The first end 64a may be fitted within or over the second passageway 60, or may be designed as a unitary piece with the second passageway 60. This arrangement allows communication between the aspiration nozzle 22 and the second passageway 60 and the throughbore 62 of the housing 50. The assembly may comprise an optional tubing clip or clips 72, seen in FIG. 5, to aid in positioning the tubing length 64 between the aspiration nozzle 22 and the second passageway 60 of the connector 56.

As illustrated particularly in FIG. 4, when fluid discharge is desired, fluid 74 flows from the discharge nozzle 20, of the handpiece 12 and through first passageway 58. The fluid 74 continues through the needle shaft 32 and exits at the needle tip 34.

Aspiration of the site is done through the through bore 62, as indicated by the arrows in FIG. 4. The bore 62 allows the aspirate (not shown) to be carried through the aspiration nozzle 22 and out the outlet 26 (seen in FIG. 1). This arrangement minimizes clogging of the aspiration pathway that potentially could occur in prior art devices. Prior art devices were not properly arranged to allow adequate removal pathways within the same assembly as one that provided fluid flow. Also, the arrangement provides for controlled and directed fluid flow, thereby reducing problems related to damage to tooth walls and the surrounding areas from spraying fluids into the root canal independent of where the needle 30 is placed within the assembly 10.

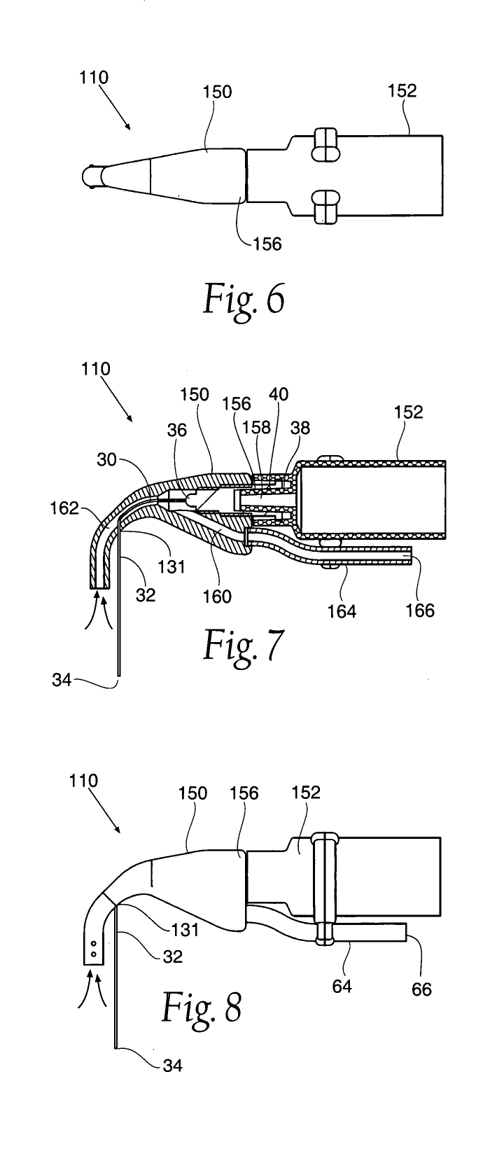

FIGS. 6-8 provide an alternate embodiment 110 of the present invention. The needle assembly 110 is similar to the assembly 10, except that the irrigation and aspiration pathways are not entirely co-axially aligned. The needle assembly 110 comprises a housing 150 and a mating section 152, similar to the assembly 10. Likewise, as stated with the assembly 10, the mating section 152 is not necessary for the present invention to function, but may be used to adapt the assembly 110 to various dental tools and provide connection means for the assembly 110 to a dental device. The mating section 152 will mate with or incorporate the LUER® lock 40. As with the previous assembly 10, the mating section 152 will be of any suitable shape or arrangement that will allow the assembly 110 to be connected to a dental device.

The housing 150 comprises a general connector area 156 that has a first passageway 158 adapted to receive the needle 30 and a second passageway 160 connected to a through bore 162 to allow a pathway for aspiration to take place with the assembly 110. The first passageway 158 preferably contains the hub member 38 which grippingly engages the needle shaft 32 to provide connection to the LUER® lock 40. The hub member 38 may be secured within the passageway 158 in similar fashions as described with respect to the assembly 10. The needle 30 is positioned outside a portion of the through bore 162 and, as shown, may in fact intersect the through bore 162 at an exit 131. Because of the preferred flexible material of the housing 150, the exit and the needle 30 will form a fluid tight connection. Additional adhesives or other structures (not shown) may be used to secure the shaft 32 of the needle 30 in close proximity to the housing 150.

The second passageway 160 of the connector 156 is adapted to receive the flexible tubing length 64 having the throughbore 66, similar to the arrangement described with respect to the assembly 10. The second passageway 160 communicates with the through bore 162 to provide aspiration means for the assembly 10 and an attached dental device. The through bore 162 is not co-axially aligned with the shaft 32 of the needle 30. The connector 156 may be arranged so that the first passageway 158 and the second passageway 160 intersect each other, or run parallel with each other. The passageway 158 may also be separately arranged from the through bore 162. The assembly 110 thus further demonstrates the utility of the present invention. Because the aspiration and irrigation pathways do not necessarily have to be positioned together or co-axial, there is more flexibility in the design of the overall needle assembly. Provided that a needle assembly allows for both aspiration and irrigation in the same assembly, with irrigation being performed through the needle and aspiration being performed externally and independently of the needle, the assembly would fall within the scope of the present invention. The irrigation needle and the aspiration passageway may be co-axially aligned, parallel paths, or other possible arrangements.

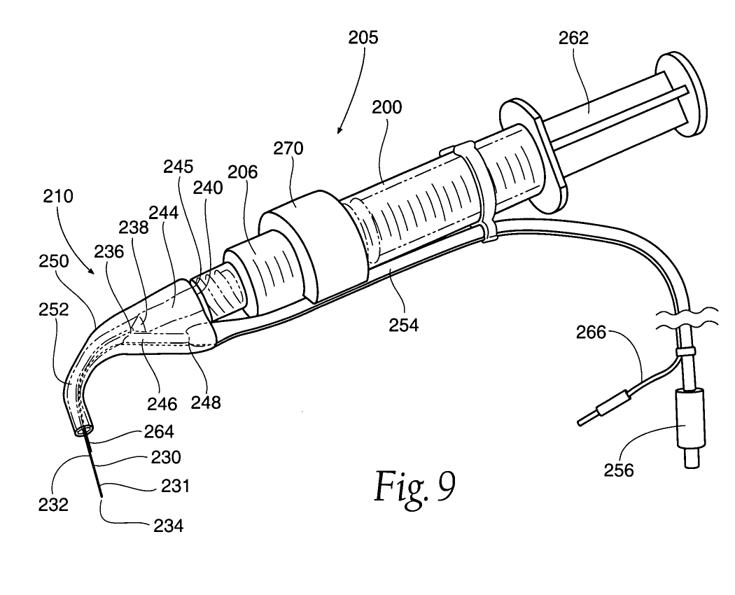

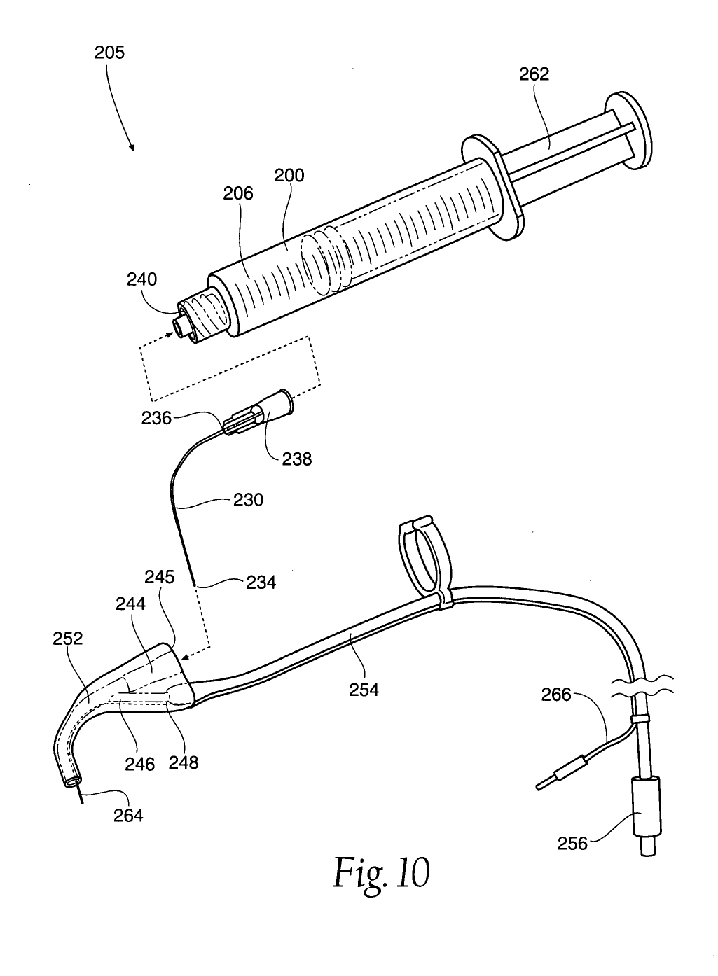

As noted above, the needle assembly according to the present invention should not be limited to use with any specific dental device, but could be adapted for use with a wide arrange of dental devices, including automatic or mechanical devices. For example, FIGS. 9-10 show a needle assembly 210 attached to a syringe 200. The assembly 210 and the syringe 200 can be considered together as a dental tool 205. As with the previous embodiments, the assembly 210 generally comprises a needle 230 and a housing 250. The needle 230 comprises a shaft 231 having a hollow bore 232. The needle has a tip portion 234 and an end attachment portion 236. The attachment portion 236 is secured within a hub apparatus 238 that is affixed to attachment means 240. The attachment means 240 is preferably an easy to attach arrangement, such as a LUER LOK®-type design, a lip tip fitting, or an eccentric fitting. The hub apparatus 238 is securely attached to the attachment portion 236 of the needle 230, possibly with the use of an adhesive. Likewise, the hub apparatus 238 is secured to the attachment means, also possibly using an adhesive material. Each of these elements could be secured together in other fashions, such as press-fitting, or the hub apparatus and the attachment means may be formed or molded as a single piece or device.

The housing 250 has a first section 244 and a second section 246. The first section 244 has an opening 245 that is designed and shaped to receive the hub apparatus 238 and the attachment means 240, with the apparatus 238 and the attachment means secured by any suitable fashion, such as adhesives, fasteners, press fit in place, or any other suitable means. The second section 246 comprises a passageway 248 that is connected to a through bore 252 located within the housing 250. The through bore 252 also preferably is in communication with the opening 245 of the first section 244, thereby allowing the shaft 231 of the needle 230 to be housed within the through bore 252.

The passageway 248 is connected to a tubing 254, preferably flexible tubing. The passageway 248 and the tubing 254 could be removable or permanently secured to one another, and each could be of any suitable length. The tubing 254 will be connected to an adaptor 256, which will allow the tubing to be connected to a suction device or evacuation source (not shown). In one preferred embodiment, the tubing 254 is secured to the adaptor 256 and is sufficiently long enough so that the dental tool 205 is easily portable and use and movement of the tool is not inhibited by the location of the aspiration source within the operating room. Thus, material will be removed through the through bore 252, the passageway 248 and the tubing 254, separately from the fluid flow from the syringe 200 to the needle 230. The adaptor 256 allows the assembly 210 to be connected to any suitable suction supply, preferably standard devices used within a normal dental office.

Referring further to FIGS. 9-10, the syringe 200 comprises an irrigation fluid reservoir 206 and a plunger 262. The plunger 262 allows the user to selectively deliver fluid to the dental site, separately from the aspiration action of the device 210. The fluid reservoir 206 allows for the dental tool 205 to work as a portable device, which gives the arrangement greater freedom over the prior art. Because the tool 205 is not concerned with connection to an external fluid reservoir, the tool 205 has a greater range than prior art devices, with less chance cords or tubes becoming tangled.

It is desirable that the irrigation and evacuation pressures are approximately balanced or that the evacuation pressure is slightly greater than the irrigation pressure to provide a net negative pressure within the tool 205. The balanced or slight negative pressure serves to help prevent caustic chemicals from passing from the root canals (not shown) into the periradicular tissue and or other tissue in the vicinity of the root apices.

Also shown connected to the tool 205 is an electrode 264, which is connected to an electrical connector or lead wire 266. The lead wire 266 is connected to a power source and a second electrode normally located at another point on the patient (not shown), as is known in the industry, to provide an electrical stimulant to promote iontophoresis. The electrical stimulant could be a current, an electrical field or electrical signal, with or without the use of an electrical current. The electrode 264 is preferably placed near the tip portion 234 or attached to the needle tip 234, thereby electrically charging the exiting fluid from the needle 230, which increases the efficacy of the fluid and may provide better irrigation. Alternatively, the needle 230 itself could be the electrode. The lead wire 266 could be arranged internally of the tubing 254, or be positioned externally of the tubing 254 and/or the tube 205 in general.

The operating parameters for an electrical device used in connection with the present invention can vary depending on the type of treatment system to be used in connection within the system. For instance, if the electrode 264 would be used to promote iontophoresis, the electric field range would be approximately 1-500,000 V/m, with a preferred field of around 1000 V/m. The current range would preferably be around approximately 100 μA-100 mA, with a preferable current being variable between about 1-50 mA. Direct or alternating current could be used in the system, with direct current being preferred.

If the electrode 264 was being used for promotion of electroporation, also known as electropermeabilization, the preferred frequency would be delivered in a range of 0-50,000 Hz, with a more preferred frequency being around 40 Hz. The preferred potential of the system would be between about 100 V-5000 V, with a current of around 5 mA. However, it is understood that any operating parameters that would be used to improve the system would fall within the scope of the present invention. Likewise, application of various signal pulse widths and duty cycles is also possible.

As stated, the evacuation tubing 254 is coupled to a vacuum source as is known in the art (not shown). Various controls may be employed to regulate the force of the vacuum source. However, it is desirable that the irrigation and evacuation pressures are approximately balanced or that the evacuation pressure is slightly greater than the irrigation pressure to provide a net negative pressure within the device 210. The balanced or slight negative pressure helps prevent caustic chemicals from passing from the dental area, such as a root canal, being treated by the device, into the sinus cavity or periradicular tissue.

The tool 205 may also include a heating element 270 for heating the solution before it is delivered through the assembly 210. Preferably, the solution would be preheated to 90° F. to 150° F., but the heating element 270 could be arranged for any temperature that will help to increase the efficacy of the solution being delivered by the tool 205 and the assembly 210.

The foregoing is considered as illustrative only of the principles of the invention. Furthermore, since numerous modifications and changes will readily occur to those skilled in the art, it is not desired to limit the invention to the exact construction and operation shown and described. While the preferred embodiment has been described, the details may be changed without departing from the invention.

I claim:

1. A needle assembly for dispensing fluids from a fluid source and for evacuating a cavity during an endodontic procedure, said needle assembly comprising:
a housing;
a surgical needle having a hollow shaft, said shaft having an inlet and an outlet, said inlet in fluid communication with said fluid source;
a first pathway located within said housing, said first pathway having an inlet and an outlet, said inlet of said first pathway containing said surgical needle inlet, said surgical needle being substantially coextensive with said first pathway;
a hub connector located in said inlet of said first pathway, said hub connector coupling said needle to said fluid source;
a second evacuation pathway located within said housing having an inlet and an outlet, said second pathway providing evacuation means for said needle assembly, said evacuation pathway being independent and external of said surgical needle.

2. The needle assembly according to claim 1 wherein said second evacuation pathway intersects said first pathway.

3. The needle assembly according to claim 1 further comprising means for providing an electrical stimulant to said fluid exiting said needle outlet.

4. The needle assembly according to claim 3 wherein said means for providing said electrical stimulant further comprises:
An electrode located near said needle outlet; and
an electrical source in electrical communication with said electrode.

5. The needle assembly according to claim 1 wherein said hub connector further comprises a LUER® lock fitting.

6. A needle assembly for dispensing fluids from a fluid source and for evacuating a cavity during an endodontic procedure, said needle assembly comprising:
a housing;
a needle having a hollow shaft for dispensing fluids, said needle having an inlet and an outlet, said inlet in fluid communication with said fluid source;
an evacuation pathway located within said housing for evacuating said cavity, said first pathway having an inlet and an outlet, said inlet positioned proximal to said needle outlet, said outlet being connectable to an evacuation device; and
a second pathway located within said housing, said second pathway having an inlet and an outlet, said needle being substantially coextensive with said second pathway.

7. The device according to claim 6, wherein said evacuation pathway intersects said second pathway, said inlet of said evacuation pathway and said outlet of said second pathway being coextensive with one another.

8. The device according to claim 7, wherein said evacuation pathway provides evacuation means exteriorly and independent of said needle, said evacuation means providing a net negative pressure for said device.

9. The device according to claim 8, further comprising means for connecting said needle to said fluid source.

10. An assembly for dispensing fluids and for evacuating a cavity during an endodontic procedure, said needle assembly comprising:
a fluid reservoir;
means for dispensing fluid from said reservoir;
a housing;
a needle having a hollow shaft for dispensing fluids, said needle having an inlet and an outlet, said inlet in communication with said fluid reservoir; and
a first evacuation pathway located within said housing for evacuating said cavity, said first pathway having an inlet and an outlet, said inlet positioned proximal to said needle outlet, said outlet being connectable to an evacuation device.

11. The assembly according to claim 10, wherein said fluid reservoir comprises a syringe.

12. The assembly according to claim 10 further comprising means for connecting said housing to said fluid reservoir.

13. The assembly according to claim 12 further comprising a second pathway located within said housing, said second pathway having an inlet and an outlet, said needle being substantially coextensive with said second pathway.

14. The assembly according to claim 13
wherein said connecting means further comprises a hub connector located in said inlet of said second pathway, said hub connector coupling said needle to said fluid source.

15. The needle assembly according to claim 14 further comprising a syringe, said syringe comprising said fluid reservoir.

16. The needle assembly according to claim 14 further comprising means for heating said fluid before entering said needle.

17. The needle assembly according to claim 10 wherein said first pathway being exteriorly arranged and independent of said needle.

18. The needle assembly according to claim 17 further comprising an electrical stimulant for said fluid exiting said needle outlet.

19. The needle assembly according to claim 18 wherein said electrical stimulant further comprises a electrode located near said needle outlet.

20. The needle assembly according to claim 19 wherein said needle comprises said electrode.

21. A method for dispensing fluids and for evacuating a cavity during an endodontic procedure, the method consisting of the steps of:
providing a fluid source;
providing an evacuation source;
connecting a needle assembly to said fluid source and said evacuation source, said needle assembly comprising:
a housing;
a needle having a hollow shaft for dispensing fluids, said needle having an inlet and an outlet, said inlet in fluid communication with said fluid source; and
a first evacuation pathway located within said housing for evacuating said cavity, said first pathway having an inlet and an outlet, said inlet positioned proximal to said needle outlet, said outlet being in communication with said evacuation source, said first evacuation pathway being independent and external of said surgical needle; and
dispensing fluid into said cavity while simultaneously evacuating said cavity.
